(19) United States
(12) Patent Application Publication
(10) Pub. No.: US 2005/0014288 A1
(43) Pub. Date: Jan. 20, 2005

(54) Motion induced electrical field stimulation

(75) Inventors: Jeffrey L. Pfohl, Durham, NC (US); Martha McLean Bolton, Chapel Hill, NC (US); Jennings Fanklin Worley III, Durham, NC (US)

Correspondence Address: Finnegan, Henderson, Farabow,;Garrett & Dunner, L.L.P., 1300 I Street, N.W., Washington, DC, 20005-3315, US

(73) Assignee: Amphora Discovery Corporation

(21) Appl. No.: 10/620,582

(22) Filed: Jul. 15, 2003

(51) Int. Cl. G01N 33/543

(52) U.S. Cl. 436/518

(57) ABSTRACT

Methods of generating a varying potential across a membrane of closed membrane structure, methods of detecting the functioning of a protein of a closed membrane structure, and methods of detecting the effects of an agent on the functioning of a protein of a closed membrane structure, by moving the structure, suspended in a fluid, through an electric field by non-ionic transport means to induce a varying potential across the membrane, are disclosed. Apparatus for generating a varying potential across a membrane of a closed membrane structure, apparatus for detecting the functioning of a protein of a closed membrane structure and apparatus for detecting the effect of an agent on the functioning of a protein of a closed membrane structure, by moving the structure, suspended in a fluid, in an electric field to induce a varying potential across the membrane, are also disclosed.

### TECHNICAL FIELD

Embodiments consistent with the invention generally relate to methods and apparatus for detecting functioning of a protein and, more particularly to methods and apparatus for detecting the functioning of a protein of a closed membrane structure.

### BACKGROUND OF THE DISCLOSURE

In medical research, measurements and analyses of biological functions have many important uses. For example, some biological structures, including living cells, include a membrane that encloses the structure and performs many functions crucial to the activity of the structure. In order to understand disease processes and develop new drugs to treat diseases, it is desirable to monitor and manipulate various characteristics of the functions of the cell membrane. One important example of these characteristics is the electrical voltage, or potential, which occurs across membranes.

The electrical potential across a biological membrane is important for many reasons. In living cells, the potential across the membrane, known as the transmembrane potential, is determined by the concentration of electrical charges, often in the form of ions, on opposite sides of the membrane. Various proteins and protein complexes are known to regulate the concentration of ions on either side of the membrane and thereby establish the transmembrane potential. The activity and functioning of membrane proteins and membrane complexes in turn may influence the transmembrane potential by altering ion movement across the membrane.

Ion “channels” are membrane proteins and protein complexes that may include an ion-selective aqueous hole, or pore, that controls the movement of ions through a biological membrane. Ion channels that are regulated, at least in part, by transmembrane potentials are generally referred to as voltage-gated ion channels.

Voltage-gated ion channels regulate the flow through the membrane of ions such as sodium ions, potassium ions, chloride ions and calcium ions. This regulation is in turn influenced by the transmembrane potential in at least two ways. First, the transmembrane potential can cause changes in the molecular configuration of the proteins forming the pore of the channel to open and close the pore. Second, the transmembrane potential directly influences the flow of ions through an open channel.

There are an increasing number of disease processes found to be associated with ion channel function. Thus, ion channels are recognized as attractive targets for therapeutic manipulation by drugs. Accordingly, there exists a need for methods and apparatus for analyzing the functioning of ion channels and the effect of agents, such as drugs, on ion channel function.

One method of studying ion channels in a cell by producing a transmembrane potential is to directly apply a voltage across the cell membrane. These voltage clamp techniques employ a rapid step in transmembrane voltage to measure the resulting ionic currents and have been used to study the electrical properties of cell membranes and membrane associated proteins. Two common contemporary examples are the patch clamp method and two-microelectrode recording methods which use very, very small glass electrodes that reside on or pierce the membrane of a single cell, respectively.

Another method of studying ion channels by inducing a transmembrane potential is to generate the transmembrane potential via application of an external electric field to the cell. The cell membrane acts as an insulator, or dielectric, and in response to the externally applied electric field, electric charges become distributed on opposite sides of the membrane. These charges generate a transmembrane potential that is defined by the strength, orientation, and location of the applied electric field with respect to the membrane. One side of the membrane of the cell will thus become more hyperpolarized while the opposite side is equally, and oppositely, more depolarized. Voltage-gated ion channels located in the hyperpolarized or depolarized region will have different protein conformational states. For many types of voltage-gated ion channels, the more hyperpolarized region of the membrane will enhance a resting, or non-activated, conformational state, while the depolarized region of the membrane enhances an inactivated state. While under these conditions, voltage-gated ion channels may also be mostly non-conducting, the two states differ in their ability to transition to an open, ion conducting state. In the hyperpolarized region, ion channels in the resting state are poised to be activated and reach an open state. However, in the depolarized region ion channels in the inactivated state are unlikely to activate or open until the protein transitions to a resting state. These transitions among states are determined by the magnitude and rate the membrane potential is changed, the type of ion channel, as well as the cell type.

When the electric field is removed, the transmembrane potential changes, equalizing or returning to an unstimulated potential. As the previously hyperpolarized region of the membrane decays to a more depolarized level, ion channels in the hyperpolarized region can become conductive, e.g., allow ions to flow through the open pore, as the protein transitions from the resting to an open state. Simultaneously, the ion channels in the depolarized region of the membrane transition from the inactivated state to the less depolarized level, however, the ion channels will remain mostly closed. Stimulating the ion channels in this way allows the ion channels to be studied using dynamic physiological voltages.

Since stimulating ion channels by an electric field requires transient induction of the transmembrane potential to activate ion channels, oscillatory electric fields have been used in the past to study ion channel phenomena. Field simulation techniques, whereby the transmembrane potential is manipulated by means of an external electric field, have been successfully employed to study the effects of pharmacological and other agents on ion channel activity in many cell types such as neuronal and muscle (skeletal and cardiac) cell types. In this method, oscillatory or pulsed applied electric fields are used to induce a transient membrane potential sufficient to activate ion channels in stationary cells. However, the transmembrane potential regulates both the ion channel activation and deactivation processes. While the ion channel activation process is rapid, occurring on the order of milliseconds, voltage-dependent inactivation is also a rapid process. Thus, in response to a transient electric field, voltage-gated ion channels remain in the activated, or open state for only a brief period of time during each cycle.

As described above, manipulation of the transmembrane potential has the potential to provide valuable insights into the operation of ion channels. In order to apply these insights to efficiently develop new and improved pharmaceutical compounds, it is desirable to measure ion channel activity and function using large scale, high throughput screening (“HTS”). However, adapting existing techniques for manipulating the transmembrane potential to measure the functioning of voltage-gated ion channels to HTS formats has been less than satisfactory.

One known method employs open-well or plate-based HTS platforms that have been developed to measure ion channel activity. Examples include apparatus developed by Molecular Devices, Corp., Inc. (Sunnyvale, Calif.), and by Vertex Pharmaceuticals Inc (San Diego, Calif.) as disclosed in WO 02/08748 A2. In the open-well or plate-based HTS systems, cells comprising voltage-gated ion channels are cultured in the bottom of an array of individual wells. Reagents containing pharmacological or other agents and probes of ion-channel activity are added to each well. Transmembrane potentials can be induced by applying an electric field to the stationary cell culture by means of a pair of electrodes positioned within each well. Ion channel activity can then be detected by, for example, fluorescence from probe molecules. In these devices, the electric field is repeatedly pulsed to force a change in the transmembrane potential which in turn can activate and deactivate ion channel proteins. Although field stimulation techniques have been adapted to array-based platforms amenable to HTS, the ability to repeatedly activate ion channels remains limited by the response time of the activation/inactivation process and the methods used to evoke ion channel specific responses in a highly precise and reliable manner.

There is a need for improved methods and apparatus for generating a varying potential across a membrane for activating ion-channels and other proteins that are amendable to HTS formats.

### SUMMARY

To address the limitations of known techniques, there is provided a method of generating a varying potential across a membrane of a closed membrane structure in which the membrane contains a first fluid, comprising suspending the structure in a second fluid, applying an electric field to the second fluid, and moving the second fluid, containing the suspended structure, through the electric field by non-ionic transport means to induce a varying potential across the membrane.

There is also provided an apparatus for generating a varying potential across a membrane of a closed membrane structure in which the membrane contains a first fluid, wherein the structure is suspended in a second fluid, comprising a fluid chamber containing the second fluid, an electric field applied to at least a part of the fluid chamber, and a non-ionic transport means for moving the suspended structure in the second fluid through the electric field to induce a varying potential across the membrane.

It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several embodiments consistent with the present invention and together with the description serve to explain the principles of the invention.

FIG. 1 is a flow diagram of an embodiment of a method consistent with the present invention for generating a varying potential across a membrane.

FIG. 2 is a schematic illustration of an apparatus consistent with the present invention for generating a varying potential across a membrane.

FIG. 3 is a schematic illustration of an apparatus consistent with the present invention for generating a varying potential across a membrane and for detecting indication by a probe.

FIG. 4 is a schematic illustration of an alternate apparatus consistent with the present invention for generating a varying potential across a membrane and for detecting indication by a probe.

FIG. 5 is a schematic illustration of another apparatus consistent with the present invention for generating a varying potential across a membrane and for detecting indication by a probe.

FIG. 6 is a schematic illustration of a further apparatus consistent with the present invention for generating a varying potential across a membrane and for detecting indication by a probe.

FIG. 7 is a diagram showing the response of N-type calcium ion channels in HEK cells to the amplitude of an applied electric field induced in a manner consistent with the methods of the invention.

FIG. 8 is a diagram showing the effect of the calcium ion channel antagonist, verapamil, on N-type calcium ion channels in HEK cells using methods consistent with the methods of the invention.

FIG. 9 is an illustration of a Cell-Chip apparatus modified in a manner consistent with the present invention to provide an electric field to a flow channel for performing assays.

FIG. 10 is a schematic of a Cell-Chip apparatus modified in a manner consistent with the present invention to provide an electric field to a flow channel for performing assays.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter as set forth in the claims should at least be construed in light of the number of reported significant digits and by applying ordinary rounding techniques.

### DETAILED DESCRIPTION

Reference will now be made in detail to the present embodiments consistent with the invention, examples of which are illustrated in the accompanying figures. Wherever possible, the same reference numbers will be used throughout the drawings to refer to the same or like parts.

In the specification and the appended claims, the singular forms “a,” “an,” and “the” include plural reference unless the context clearly dictates otherwise.

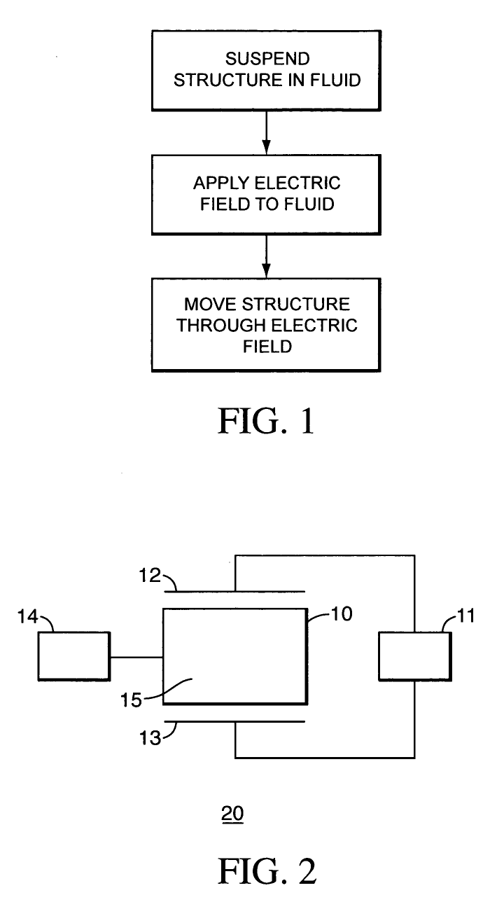

Consistent with the invention, one embodiment provides a method of generating a varying potential across a membrane of a closed membrane structure in which the membrane contains a first fluid. The method comprises suspending the structure in a second fluid, applying an electric field to the second fluid, and moving the second fluid through the electric field by non-ionic transport means to induce a varying potential across the membrane. A flow diagram of an embodiment of a method for generating a varying potential across a membrane is provided in FIG. 1.

A characteristic feature of membranes is the ability to maintain charge separation on opposites sides of the membrane; that is, the membrane is a dielectric. In certain embodiments, the membrane may be a biological membrane, such as, a plasma membrane of a cell, a membrane of an intracellular organelle, or a viral envelope. Examples of plasma membranes include plasma membranes from animal cells, insect cells, bacterial cells, and plant cells. Examples of membranes of intracellular organelles include membranes of Golgi apparatus, lysosomes, nuclear envelopes, mitochondria, endoplasmic reticulum, and chloroplasts. In other embodiments, the membrane may be an artificial membrane such as a lipid bilayer or vesicle. The artificial membrane may comprise, in whole or in part, synthetic or purified components, and components or fragments of biological membranes.

A closed membrane structure refers to the closed membrane and the internal fluid which the membrane encloses. In certain embodiments, the closed membrane structure is a naturally occurring biological structure, such as a cell. The cell may be an appropriate cell selected according to the subject of the investigation or assay. In certain embodiments, the cell may be from a cell line used in biotechnology and medical investigations. Examples of cells that may be used in the methods and apparatus of the invention include, for example, CHO-K1 cells, HEK-293 cells, RIN cells, and other appropriate excitable and non-excitable cells. The cells may be grown, harvested, and suspended in a suitable fluid by any appropriate biotechnology method.

In certain embodiments, the closed membrane structure may include more than one closed membrane structure. For example, where the closed membrane structure comprises a cell, the cell may comprise internal organelles comprising closed membrane structures. Similarly, in certain embodiments of the invention, vesicles may be produced to enclose other closed membrane structures. In other embodiments, the structure is an artificial or synthesized structure such as a vesicle.

In certain embodiments of the invention, the fluid contained by the membrane is an electrically conductive fluid. In embodiments where the structure is a cell, the enclosed fluid may be the intracellular fluid, or cytoplasm. In other embodiments, where the structure is an artificial structure, the enclosed fluid may be an appropriate electrically conductive fluid that maintains the integrity of the vesicle in the surrounding external fluid.

In embodiments consistent with the invention, closed membrane structures may be suspended in a fluid. In certain embodiments, the fluid in which the membranes are suspended may be any electrically conductive fluid that maintains the structural integrity of the closed membrane structure. In certain embodiments, the fluid may be a physiological buffer solution, such as Bicarbonate, TRIS, HEPES, and MOPS.

The fluids in which the structures are suspended may further include a certain ionic content, and/or osmolarity. The fluid may further include components that may impact the motion and rotation of the suspended structures. For example, the fluid may include particulates such as silica or polymeric beads that may either increase or decrease the rotation, turning, and translation of the suspended structures. The fluid may include compounds that increase or decrease the rotation, turning, and translation of the suspended structures by modifying the viscosity of the fluid, including, for example, polymers, Optiprep, and sucrose. The fluid may also include compounds that minimize adhesion of the suspended structures to surfaces, to other suspended structures, and to other constituents, including, for example, surfactants, bovine serum albumin, and serum. In certain embodiments, components added to the fluid may impact the rotation, turning, and translation of the suspended structures by modifying the shape of the suspended structures.

In general, when a dielectric structure suspended in a fluid is exposed to an external electric field, a potential across the structure will be induced. For a spherical dielectric of radius a, the induced transmembrane potential can be minimally expressed as Equation 1:
Δφ=1.5 a E cose θ
where E is the applied electric field strength and the angle, θ, is between the field line and the point of interest on the structure. Thus, for a spherical dielectric, the potential induced by an electric field will vary spatially over the surface of the dielectric.

For a stationary dielectric structure in an electric field, the potential at any point on the surface of the structure will be constant in time. When the dielectric is moved in the external electric field, the induced potential at any point on the surface of the dielectric will change according to Equation 1. When the motion of the dielectric is parallel to the direction of the electric field, only the magnitude, and not the polarity, of the induced potential at any point on the surface will change. When the structure turns, rotates, or translates, thus changing its spatial relationship in the electric field, both the amplitude and the polarity of the induced potential may change for a specific, local membrane area. Motion of dielectric structures, and by analogy, closed membrane structures, in an electric field will induce a potential that, for any point on the surface of the dielectric or membrane, changes in amplitude and polarity with time.

In embodiments consistent with the invention, the closed membrane structures may be moved in the fluid by one or more of several methods. In certain embodiments, the fluid may be moved so as to cause the suspended structures to be moved.

For example, in certain embodiments, the closed membrane structures may be moved by differential pressure, mechanical agitation, gradients, gravitation, or sonication. Moving the closed membrane structures causes the structures to move parallel, perpendicular, and/or at an angle to the direction of the electric field, e.g., translate, and to turn or rotate in the electric field. In certain embodiments, translation, rotation, and turning of the closed membrane structures in the electric field may be caused by, for example, fluid turbulence, differential viscous drag, collision with the walls of the fluid chamber, collision with other closed membrane structures, or collision with other particulates suspended in the fluid. However, movement of the closed membrane structures by ionic transport means such as electroosmosis and electrokinesis may interfere with the interaction of the applied electric field and the suspended structures. Thus, ionic transport mechanisms form no part of the present invention.

In other embodiments, the suspended structures may be moved within the fluid. For example, in certain embodiments, a fluid chamber containing the suspended structures in the fluid may be moved by mechanical means. In other embodiments, the suspended membrane structures may be dropped through the fluid.

Consistent with the invention, another embodiment provides a method of detecting the functioning of a protein of a closed membrane structure in which the membrane contains a first fluid comprising suspending the structure in a second fluid, applying an electric field to the second fluid, moving the second fluid, containing the suspended structure, through the electric field by non-ionic transport to induce a varying potential across local sections of the membrane sufficient to cause a change in the functioning of the protein, and detecting indication by a probe, wherein the probe indication is produced in response to the change in the functioning of the protein.

Proteins include oligopeptides and polypeptides. A peptide refers to any compound produced by amide formation between a carboxyl group of one amino acid and an amino group of another amino acid. An oligopeptide refers to a peptide with no more than 10 to 20 amino acid groups. A polypeptide refers to a peptide with more than 10 to 20 amino acid groups. Proteins may include naturally occurring proteins, non-naturally occurring proteins which include one or more synthetic peptides or modified naturally occurring peptides, and synthetic proteins.

Certain proteins may be associated with cell membranes. These membrane proteins may be situated on the exterior or interior expanse of the membrane, or may extend through the membrane, such as a transmembrane protein. Membrane proteins may be single proteins, or may be protein complexes comprising multiple proteins. The multiple proteins of a protein complex may define multiple domains on the exterior and interior of the membrane. The sub-unit proteins of a protein complex may comprise the same or different sub-unit proteins. The sub-unit proteins of a protein complex may be linked by amino acid sequences.

Transmembrane proteins or protein complexes include at least one transmembrane domain, at least one extracellular domain, and at least one intracellular domain. Where there are two or more transmembrane domains, the transmembrane domains may be linked by amino acid sequences. Transmembrane proteins include, for example, GPCRs/7TMs, transmembrane receptors, ion channels, and transporter proteins. Transmembrane proteins may be single proteins, or may include more than one protein associated into a complex. Individual protein subunits comprising a protein complex may be the same or different proteins.

The protein may be an endogenously expressed protein or protein complex, or an exogenously expressed protein or protein complex. The term endogenous protein refers to a protein that is produced as a natural consequence of cell function. An endogenous protein may be expressed at natural levels or the expression of the endogenous protein may be amplified by known biotechnology methods. In other embodiments, the protein may be an exogenous protein. The term exogenous refers to a protein that is provided to the cell, such as, for example, by transient transfection biotechnology protocols using common reagents or viral delivery. For example, protein expression may be regulated through a choice of expression vectors, inducible-expression vectors, viral expression systems, or chemically through the use of sodium butyrate. For some proteins, an increase in the number of proteins at, for example, the cell surface, can be achieved by co-expression expression of an additional protein subunit or of a trafficking factor. In certain embodiments, the closed membrane structure may comprise endogenously expressed and exogenously expressed proteins.

Without being limited by theory, the function and activity of proteins may be determined by the protein conformation. The conformation of proteins may be determined by the interaction of the protein with the local electrochemical environment, substrates, reactants, ligands, temperature and, for certain proteins, the transmembrane potential. Through interdependent biochemical and physiological pathways and mechanisms, the function and activity of proteins can be indirect as well as direct.

Certain membrane proteins may be affected or modulated by the transmembrane potential. The transmembrane potential in the local region of the membrane where the protein is situated may induce a change in the conformation of the membrane protein. The change in conformation may influence the function and/or activity of the membrane protein.

Ion channels, including voltage-gated ion channels, ligand-gated ion channels, mechanically-gated ion channels, and non-voltage gated ion channels may be affected by, or modulated directly or indirectly by the transmembrane potential. Examples of voltage-gated ion channels include sodium ion channels, potassium ion channels, chloride channels, and calcium ion channels. Examples of ligand-gated channels having distinct ligand binding, ion selectivity, and conductance properties include neurotransmitter-gated ion channels. For example, the acetylcholine-, serotonin-, or glutamate-gated channels, at excitatory nerve synapses, regulate the passage of cations through the membrane, and the glycine and γ-aminobutyric acid-gated ion channels, at inhibitory nerve synapses, regulate the passage of anions through the membrane. Active ion transport proteins and protein complexes directly or indirectly coupled to ATP phosphorylation energy pathways may also be affected by or modulated by the transmembrane potential. Other transmembrane proteins not directly associated with ion transduction, such as kinases, phosphatases, and proteases, may also be affected or modulated by the transmembrane potential.

In addition to membrane proteins, other proteins may be affected by the transmembrane potential. Non-membrane proteins may be affected by biochemical pathways involving membrane proteins that are influenced by the transmembrane potential. For example, increased intracellular ion levels may influence the expression of intracellular protein levels through RNA directed synthesis.

By means of the highly interconnected nature of cellular processes, events affected by the transmembrane potential may cascade to impact a wide range of intracellular phenomena. Thus, the transmembrane potential may affect the functioning of other biological compounds, complexes or structures. For example, the transmembrane potential may influence the activity of a membrane protein that may in turn influence the activity or functioning of RNA synthesis or DNA expression.

The functioning of proteins can be determined by detecting indication by a probe. A probe may produce a response or indication to a change in the functioning of the protein induced by the transmembrane potential. The probe may also produce an indication in response to an appropriate characteristic of a compound including, for example, the expression of a compound, the concentration of a compound, the presence of a compound, the translocation of a compound, the flux of a compound, the conformation of a compound, the activity of a compound, the environment of a compound, and the structure of a compound. The term probe generally may include any atom or molecule that is detectable or that may produce a detectable indication. In certain embodiments, the probe may include more than one atom or molecule that may or may not interact with each other. Moreover, in certain embodiments, the probe may produce more than one indication.

In certain embodiments the probe indication comprises the absorption or emission of electromagnetic radiation, including fluorescence, and luminescence. In other embodiments, the probe indication comprises nuclear radiation.

A fluorescence probe is capable of absorbing energy and emitting at least some fraction of the absorbed energy as radiation over time. Fluorescence compounds include discrete compounds, molecules, naturally occurring proteins and macromolecular complexes or mixtures of fluorescent and non-fluorescent compounds or molecules.

A luminescence probe refers to a compound capable of absorbing energy, such as electrical, e.g., electro-luminescence; chemical, e.g., chemi-luminescence; or acoustic energy; and then emitting at least some fraction of the energy as radiation over time. Fluorescence compounds include discrete compounds, molecules, bioluminescent proteins, and macro-molecular complexes or mixtures of luminescent and non-luminescent compounds or molecules that act to cause the emission of radiation. In still other embodiments, the probe indication may be electrical, chemical, or electrochemical phenomena.

In certain embodiments, the probe is sensitive to ion channel function and activity. Such probes include fluorescence probes for detecting ions such as, for example, sodium, potassium, calcium, chloride, thallium, rubidium, lithium, hydrogen, bicarbonates, and nitrates. Other probes sensitive to ion channel function and activity include fluorescent membrane potential probes such as oxonol, rhodamine derivative dyes, styryl derivative dyes, fluorescence resonance energy transfer (“FRET”)-based voltage sensors, GFP-type mutants, and photoproteins. FRET refers to fluorescence resonance energy transfer including energy transfer processes that occur between two fluorescent components, a fluorescent component and a non-fluorescent component, a luminescent component and a fluorescent component and a luminescent component with a non-fluorescent component.

The probe may be situated in any location suitable for producing a detectable indication in response to a change in functioning of a protein. In certain embodiments, the probe may be situated on the exterior expanse of the closed membrane structure, on the interior expanse of the closed membrane structure, or within the surface of the closed membrane structure. In other embodiments, the probe may be situated in the fluid surrounding the closed membrane structure, or in the fluid within the closed membrane structure. In still other embodiments, the probe may be situated on physical surfaces such as, for example, the sides of the chamber holding the suspended structures, or a platform surface in which the suspended structures are placed after passing through an applied electric field. In certain embodiments, the probe may be ionically or covalently attached to other molecules including the protein being assayed.

The probe may be introduced into the system including the suspended structure, the fluid, and the apparatus by any appropriate biotechnology technique.

Indication by a probe may be detected by a variety of appropriate means and methods. For measuring the effects of a varying transmembrane potential on the functioning of ion channels, certain methods for detecting include, for example, FRET-based voltage sensors, electrochromic transmembrane potential dyes, transmembrane potential redistribution dyes, extracellular electrodes, field effect transistors, radioactive and non-radioactive flux assays, ion sensitive fluorescent or luminescent dyes, ion sensitive fluorescent or luminescent proteins, the expression of endogenous proteins or the use of reporter genes or molecules. Instrumentation for measuring probe indication by optical methods include microscopes and plate readers as well as other devices designed to deliver excitation light and detect emission light. The instrumentation may include a variety of techniques for manipulating the indication by the probe, such as, for example ratiometric techniques, time-resolved techniques, spectral dispersion techniques, and polarization techniques.

In other embodiments, instrumentation for detecting indication by a probe may include radiometers, electrodes, chemical analysis instrumentation, atomic absorption and optical measurement devices and other instrumentation known to those skilled in the art. In certain embodiments, more than one detector may be used, and the multiple detectors may employ different detection systems. The detectors may measure the indication of a single or multiple probes or different characteristics of the indication of a single or multiple probes.

Consistent with the invention, another embodiment includes methods and apparatus for detecting the effect of an agent on the functioning of a protein of a closed membrane structure. An agent refers to a naturally occurring or synthetic compound that may interact to cause a change in the function, activity, or expression of phenomena, such as the functioning of a protein. For example, the agent may affect the conformation of a protein or protein complex that regulates the flux of ions across membranes. The agent may be a ligand that binds to the protein. The agent may act as agonist to increase the activity of the protein, as an antagonist to decrease the activity of the protein, or act as an inverse agonist that binds to a protein receptor that modulates the ion flux. The agent may affect the extracellular or intracellular concentration of ions and thereby modulate the flux of ions through the ion channel. The agent may affect the expression of ion channels by, for example, modulating the synthesis or translocation of ion channel proteins. The agent may affect ion transporters that utilize cellular ATP energy. The agent may affect the target ion channel directly or indirectly. The agent may affect one or more ion channels or have one or more affects on cellular function. The agent may affect or modulate the ion channel by acting from the outside of the membrane, by acting from the inside of the membrane, or by intercalation into and acting within the membrane. The agent may comprise one or more agents.

In certain embodiments, the agent may be a pharmacological agent, a compound considered to be a potential pharmacological agent, or a compound to be screened or assayed for activity as a pharmacological agent. A pharmacological agent refers to an agent that is known to be, may be, or is determined to be useful in the treatment of a disease or disorder.

In certain embodiments of the invention, apparatus for generating a varying potential across a membrane of a closed membrane structure comprise a fluid chamber, a fluid comprising the suspended structure, an electric field applied to at least part of the fluid chamber, and a non-ionic transport means for moving the suspended structure in the fluid through the electric field to induce a varying potential across the membrane.

One embodiment of an apparatus consistent with the invention is schematically illustrated in FIG. 2. As shown, system 20 comprises a fluid chamber 10 containing a fluid 15. Fluid chamber 10 may be of any appropriate dimension capable of holding a suspension of closed membrane structures, such as for example, a cuvette, a pipette, a channel of a planar substrate, or a capillary tube. In certain embodiments, the dimensions of fluid chamber 10 are such that when fluid 15 and/or structures are moved, the structures are capable of rotating, turning, and/or being translated. In certain embodiments, the dimensions of fluid chamber 10 are selected to modulate the amount of time that the suspended structures remain exposed to an applied electric field. This may be accomplished by, for example, increasing the dimensions of fluid chamber 10, such as, for example, increasing the volume of the cuvette, or in a channel or capillary configuration, increasing the length of the channel or capillary. The apparatus further includes a transport mechanism 14 for moving fluid 15 in fluid chamber 10.

An electric field is provided by at least two electrodes 12, 13 positioned to provide an electric field gradient across fluid chamber 10. Electrodes 12, 13 are biased by voltage source 11, such that, for example, electrode 12 is positive and electrode 13 is ground. Biased electrodes 12, 13 may be parallel or non-parallel with respect to each other or the fluid chamber 10. More than one pair of electrodes 12, 13 may be used to provide a desired electric field to the suspended structures in fluid 15 of fluid chamber 10. Electrodes 12, 13 may provide an electric field across the entire fluid chamber 10, or part of fluid chamber 10. Depending on specific requirements, electrodes 12, 13 may be external to fluid chamber 10, and in other embodiments electrodes 12, 13 may be within fluid chamber 10, and/or may be contacting fluid 15 in fluid chamber 10. For example, electrodes 12, 13 may be attached or patterned onto the sides of fluid chamber 10. Electrodes 12, 13 may be of any appropriate dimension and form, such as, for example, a wire, multiple wires, a plate, or an array of plates. Electrodes 12, 13 may comprise any electrically conductive material such as, for example, copper, silver, or gold. In certain embodiments, electrodes 12, 13 may be coated with a non-conductive material, such as a polymeric film.

The separation between electrodes 12, 13 may be any amount to form a varying transmembrane potential across the membranes of structures suspended in fluid 15. The separation between the electrodes may be determined by the geometry of the fluid chamber and by the maximum amplitude of the desired applied electric field.

In certain embodiments, the separation between the electrodes may range from 0.001 cm to 100 cm. While in many embodiments changing the amplitude of the electric field may be most easily accomplished by changing the voltage applied to electrodes 12, 13 by the voltage source 11, in certain embodiments it may be desirable to change the separation between electrodes 12, 13 to change the amplitude of the applied electric field.

The amplitude of the electric field may be any amplitude that can generate a varying potential across a membrane of a closed membrane structure. In certain embodiments, the amplitude of the applied electric field can range from 0.1 V/cm to>600 V/cm.

In certain embodiments, the amplitude of the electric field is uniform within the region of fluid chamber 10 exposed to the electric field. For example, a spatially uniform electric field may be generated by applying a voltage bias between two parallel plates. In other embodiments, the amplitude of the electric field may be spatially non-uniform, or spatially vary within the region of fluid chamber 10 exposed to the electric field. A spatially non-uniform electric field may be produced, for example, by using multiple electrodes, or by having electrodes 12, 13 be non-parallel. Each of a plurality of electrodes may have a different bias voltage to produce a different electric field.

The electric field generated between electrodes 12, 13 may have a variety of temporal characteristics to induce a desired varying potential across a membrane of a closed membrane structure. In certain embodiments, the electric field generated between electrodes 12, 13 will be constant in time as characterized by a DC waveform. In other embodiments, the temporal characteristics of the electric field may be discontinuous. Examples of discontinuous waveforms include pulsed waveforms and stepped waveforms.

In other embodiments, the temporal characteristics of the electric field may vary continuously with time. In certain embodiments, the continuously varying electric field may be characterized by a repetitive waveform such as an AC waveform or a saw tooth waveform in which the magnitude of the waveform may or may not change polarity. In other embodiments, the temporally varying waveform may be characterized by an amplitude that varies in a defined fashion, such as a ramped waveform.

In certain embodiments the electric field may be spatially and temporally uniform between electrodes 12, 13. In other embodiments the electric field may vary both spatially and temporally between electrodes 12, 13. In both embodiments, where the apparatus includes a plurality of electrodes, any pair of electrodes may produce a spatially uniform electric field, a spatially inhomogeneous electric field, a temporally constant electric field, a temporally varying electric field, or any combination thereof.

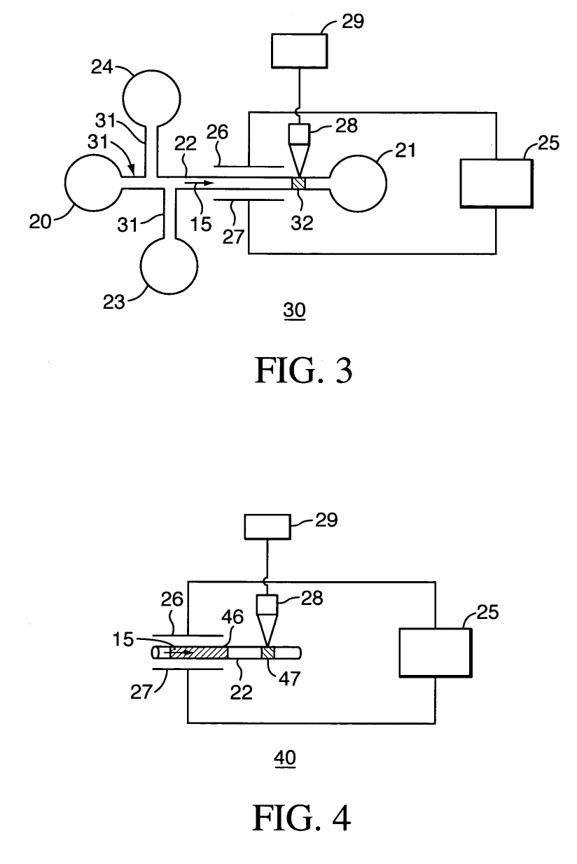

Another embodiment of an apparatus consistent with the invention is schematically illustrated in FIG. 3. FIG. 3 shows a system 30 formed on a planar substrate, such as a microfluidic apparatus. The system comprises a flow channel 22 with one or more reservoirs 20, 23, 24 connected to flow channel 22 by intersecting channels 31. As shown, reservoirs 20, 23, 24 may contain fluid with suspended structures, probes, agents, fluid compositions, fluid agents, buffer solutions, or other fluids. As shown, a reservoir 21 receives fluid from flow channel 22 and in certain embodiments is a receiving reservoir. The dimensions of flow channel 22 and flow characteristics are sufficient to enable suspended structures to rotate, turn or translate as the structures pass through the region of flow channel 22 exposed to the electric field. In certain embodiments, the cross-sectional dimensions of flow channel 22 are approximately 25 μm×100 μm. Although flow channel 22 is shown as being straight, it will be appreciated that other geometries may be used. For example, in certain embodiments, serpentine channel geometries may be used, for example, to increase the time the suspended structures are exposed to the electric field or to control the extent to which the suspended structures rotate, turn, or translate. The length of flow channel 22 may be any appropriate length sufficient to expose the suspended structures to the electric field for a desired time. In certain embodiments the length of the region of flow channel 22 exposed to the electric field is about 20 mm to 100 mm.

An electric field is applied to at least a region of flow channel 22 by electrodes 26, 27 which are electrically coupled to a power supply 25. In the embodiment shown in FIG. 3 electrodes 26, 27 are oriented parallel to fluid channel 22 and the direction of fluid flow. In other embodiments, electrodes 26, 27 may be oriented perpendicular to the direction of fluid flow, or at an angle to the direction of fluid flow. Electrodes 26, 27 may be in electrical contact with fluid 15 in which case electrodes 26, 27 may be placed within the reservoirs, such as, for example, in reservoirs 21 and 23. A region of flow channel 22 may be a detection region 32 where indication from a probe may be detected by detector 28 connected to detection system 29. Detection region 32 may be located within the region of the applied electric field or external to the region of the applied electric field.

In certain embodiments of an apparatus of FIG. 3, a mechanism (not shown) is provided for causing fluid 15 to flow through flow channel 22. For example, the moving mechanism may include an integrated microfluidic structure, having micropumps and microvalves, or external elements such as pumps and switching valves for the pumping and directing the various fluids through the device. In other embodiments, a means for moving may include vacuum or pressure driven devices. In certain embodiments, hydrostatic, wicking, and capillary force may be used to move the fluid. Electroosmotic and electrokinetic fluid transport means are specifically excluded. The means for moving the suspended structures may be external to flow channel 22, or may be integrated into the platform comprising flow channel 22.

Fluid transport within fluid channel 22 may be continuous or discontinuous. A continuous flow refers to an unbroken or contiguous stream of a particular fluid. For example, a continuous flow may include a constant fluid flow at a set velocity, or a fluid flow which includes pauses in the flow rate, but does not interrupt the flow stream. A discontinuous flow refers to flow of a discontinuous stream comprising more than one fluid. For example, the stream may comprise repeating segments comprising a segment of suspended structures in a fluid, a buffer segment, a suspended structure segment including an agent, followed by another buffer segment.

The rate of fluid transport within fluid channel 22 may be an appropriate rate to cause the suspended structures to induce a varying transmembrane potential while passing through the electric field. The rate of fluid transport may affect the rate of rotation, turning, and/or translation of the suspended structures. The necessary rate of fluid transport to induce a varying transmembrane potential can depend on a number of factors such as the dimensions of the fluid chamber, the density of the suspended structures, the dimensions of the suspended structures, the presence and density of particulates in fluid channel 22, and the concentration of viscosity modifiers in the fluid. The rate of fluid transport may also be determined by the desired duration of time to expose the suspended structures to the electric field and/or agents.

Another embodiment of apparatus consistent with the invention is schematically illustrated in FIG. 4. As shown therein, a system 40 includes a flow channel 22 comprising, for example, a pipette, micropipette, or capillary tube. The passage of fluid 15 through flow channel 22 may be continuous or discontinuous. In certain embodiments, the apparatus may further include means for adding suspended structures to the fluid, and adding other fluids comprising, for example, agents. Flow channel 22 includes a region 46 exposed to the electric field and a detection region 47. In certain embodiments, detection region 47 is within the region exposed to electric field 46, whereas in other applications detection occurs external to flow channel 22. As in system 30 of FIG. 3, electrodes 26, 27 may be oriented parallel to the direction of fluid flow, perpendicular to the direction of fluid flow, or at an angle to the direction of fluid flow. Electrodes 26, 27 are connected to a voltage source 25. Indication of a probe in detection region 47 may be monitored by detector 28 connected to detection system 29.

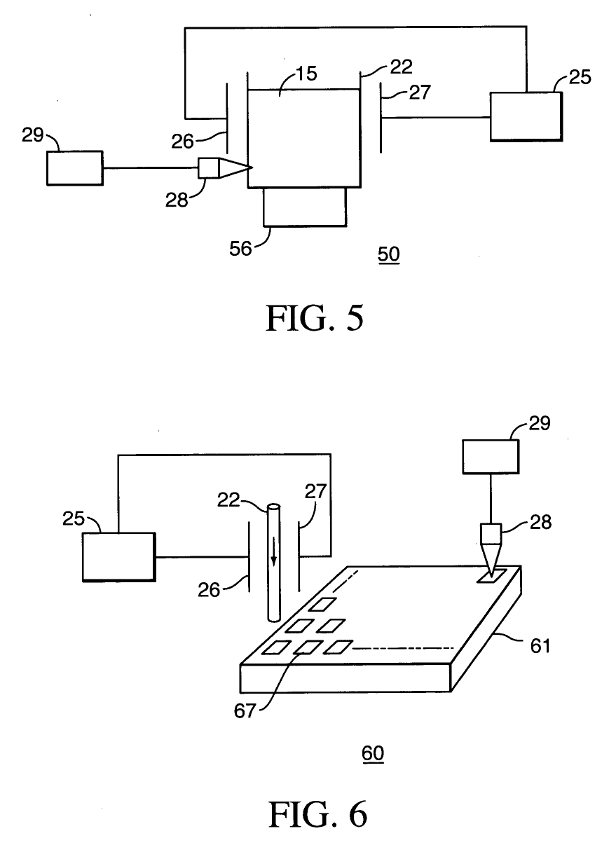

Another embodiment of the apparatus consistent with the invention is schematically illustrated in FIG. 5. As shown therein, a system 50 comprises fluid chamber 22 characterized by the ability to hold a large volume of fluid 15, such as, for example, a beaker, tube, or cuvette. The apparatus includes a component 56 for moving fluid 15 and/or suspended structures in fluid 15. In certain embodiments, component 56 may comprise a device such as a spinner placed within fluid chamber 22 to mechanically induce movement of fluid 15. In other embodiments, component 56 may comprise a heater to thermally induce movement of fluid 15. Component 56 may comprise a sonicator to sonically induce agitation of structures within fluid 15. In yet another embodiment, component 56 may induce movement either continuously or discontinuously. In another embodiment, suspended structures may move through the fluid using gravity.

System 50 includes electrodes 26, 27 connected to voltage source 25. Electrodes 26, 27 may be positioned and oriented so as to expose at least a part of fluid chamber 22 containing suspended structures to an electric field. Electrodes 26, 27 may be positioned external to fluid chamber 22 or internal to fluid chamber 22 and may be in physical contact with fluid 15 in fluid chamber 22. In certain embodiments, an apparatus as illustrated in FIG. 5 includes a detector 28 connected to detection system 29. Detector 28 may be positioned external to fluid chamber 22, or, in other embodiments, detector 28 may be positioned within fluid chamber 22.

Another embodiment consistent with the invention is schematically illustrated in FIG. 6. As shown therein, a system 60 comprises a vertically oriented fluid chamber 22 positioned over a planar platform 61 comprising multiple sample wells 67. Planar platform 61 comprising multiple sample wells 67 is referred to as a plate reader. Fluid chamber 22 may comprise, for example, a pipette, micropipette, or capillary tube. Fluid chamber 22 is positioned to enable suspended structures in a fluid to pass through an electric field provided by electrodes 26, 27 connected to voltage source 25. Suspended structures may be moved through the electric field by, for example, gravity and differential fluid pressure. After passing through the electric field, the suspended structures are deposited in wells 67.

In certain embodiments electrodes 26, 27 may be positioned external to fluid chamber 22, internal to fluid chamber 22, or on the external or internal walls of fluid chamber 22. In certain embodiments, fluid chamber 22 may be moved with respect to platform 61, and in other embodiments, platform 61 may be moved with respect to fluid chamber 22. Platform 61 may include a multiplicity of wells to enable detection of indication by a probe. The material comprising platform 61 may be any suitable material including, for example, silica, silicon, metals, and polymers. Indication by a probe may be detected during or shortly after deposition of a sample on a well, or indication may be detected external to the deposition of a sample from fluid chamber 22. In certain embodiments, detector 28 connected to detection system 29, monitors indication by a probe within individual or multiple wells 67. In certain embodiments, platform 61 may comprise an integral detector 28 and detection system 29 formed, for example, by lithographic techniques known in the semiconductor industry.

Although the examples present apparatus comprising single systems, it will be appreciated that certain embodiments of the invention include apparatus comprising a plurality of such systems. Apparatus comprising a plurality of systems enable highly parallel and/or serial analysis compatible with high throughput screening. For example, in a serial system, membranes suspended in a fluid may sequentially pass through multiple electric fields, detection regions, and/or regions where various agents are present. In other systems, membranes suspended in a fluid may pass through multiple fluid chambers in parallel such that the membranes passing through each of the multiple fluid chambers is exposed to a different electric field, a different agent and/or have a different detection region. In still other systems, a combination of serial and parallel processing as described above may be employed.

### EXAMPLE

### Assay Using Modified Cell-Chip

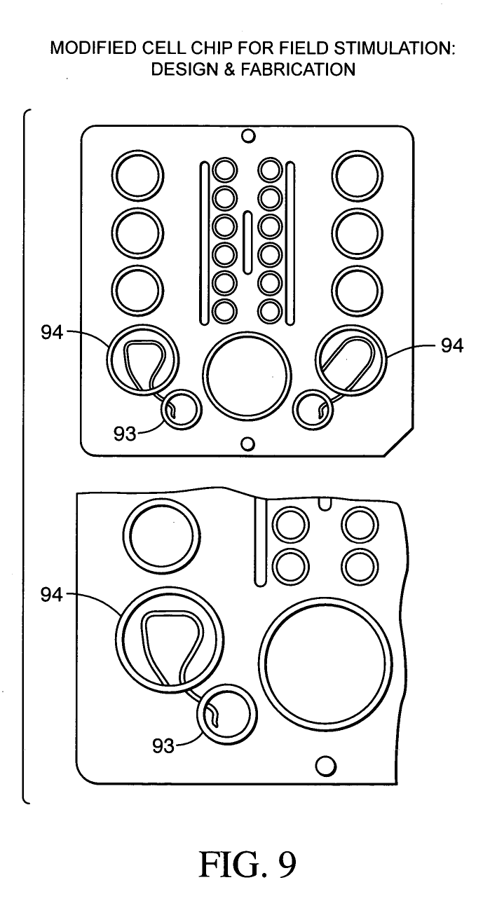

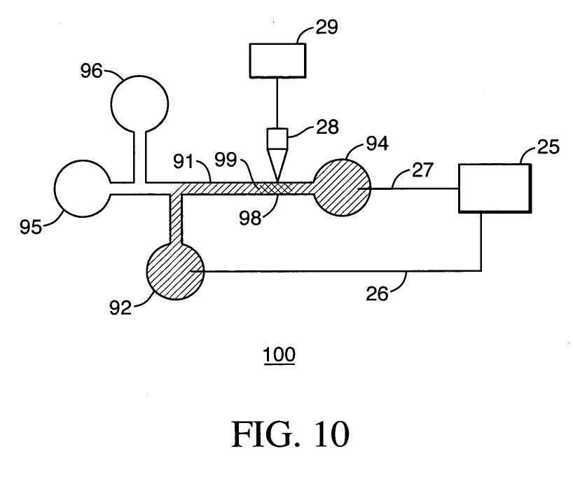

A detailed view of a system consistent with the invention is presented in FIG. 9 and FIG. 10. As shown therein, a Cell-Chip (Caliper Technologies, Mountain View, Calif., antagonist format 418D) was modified to include electrical connection to the fluid channels. The Cell-Chip includes a planar quartz glass substrate comprising multiple fluid channels over which a plastic plate comprising multiple fluid wells is positioned.

As shown in FIG. 10, system 100 comprises a fluid chamber 91 fluidly connecting a cell well 95, a sipper/antagonist well 96, an agonist well 92, and a waste well 94. Cell well 95 contains cells suspended in a fluid which are transported through an electric field 99 and a detection window 98 to waste well 94. Agents may be added to the suspended cells from agonist well 92 and sipper/antagonist well 96. Oppositely biased electrodes are positioned in agonist well 92 and waste well 94 in electrical contact with the fluid to generate electric field 99 (diagonals) in fluid channel 91 connecting agonist well 92 and waste well 94. Fluid transport from cell well 95, agonist well 92, and sipper/antagonist well 96 to waste well 94 is accomplished by pressure.

To construct the apparatus of FIG. 10, the wells of the Cell-Chip were modified to accommodate electrodes. Small holes were made in the plastic wall of an electrode well 93 (not exposed to experimental chamber) and waste well 94. A copper or silver wire was placed in each hole to provide electrical connection to the solutions to be placed in each well. The wires and well walls were sealed with silicone.

A Caliper Personality Module was adapted to accommodate stirrers to enable suspension of cells within the wells of the Cell-Chip. The Personality Module comprises two parts. The two parts were placed on either side of the Cell-Chip previously described and the assembly tightly held together to form a lab-on-chip microfluidic system. The bottom platform of the Personality Module was modified to incorporate stirrer mechanisms such that when assembled, stirrer mechanisms were positioned under cell well 95. The bottom platform was machined to accommodate four Mini-P stirrer mechanisms (VarioMag USA, Daytona Beach, FL) and associated cabling, which was connected to a Model 40S power supply (VarioMag USA).

The following protocol was used to load cells with fluorescent probes. A solution of Fluo-4 AM fluorescent probe was prepared by mixing 30 μL of 1 mM Fluo-4 AM (Molecular Probes, Inc.) in DMSO (Sigma Chemical Company) and 30 μL of 10% Pluronic F-127 (Sigma Chemical Company) in DMSO (w/v). Separately, a solution of Fura-Red AM (Molecular Probes, Inc.) was prepared by mixing 30 μL of 1 mM Fura-Red AM and 30 μL of 10% Pluronic F-127. The Fluo-4 AM and Fura-Red AM solutions were combined with 10 mL of Loading Buffer (130 mM NaCl, 5 mM KCl, 10 mM HEPES, 10 mM glucose, 2 mM CaCl₂, 1 mM MgCl₂; pH 7.4) and 20 μL of 0.5 M Probenecid (Sigma) was then added to form the fluorescent probe dye solution.

HEK-293 cells stably expressing human N-type calcium channels were grown in DMEM, 10% fetal bovine serum, 1% penstrep and 0.5 mg/ml G418. The HEK-293 cells were gently washed with 10 mL of PBS (Physiological Buffered Saline, without MgCl₂ and CaCl₂) (Clontech). To incorporate the fluorescent probe into the washed cells, 10 mL of the probe dye solution was added to the cells and the cells incubated for 2 hours at room temperature.

Cells containing the fluorescent probe dye were harvested by first transferring the cells to a 15 mL conical tube and centrifuged at 1,400 rpm (500 G) for 2 minutes. The resulting cell pellet was re-suspended in 10 mL of Wash Buffer (130 mM NaCl, 5 mM KCl, 10 mM HEPES, 10 mM glucose without CaCl₂ and MgCl₂; pH 7.4), 2 mM EDTA, 10% water, 0.1% Pluronic F-68, and 1 mM Probenecid. The suspension was centrifuged at 1,400 rpm (500 G) for 2 minutes. The resulting cell pellet was re-suspended in 5 mL of 40% OptiPrep/Loading Buffer (Axis Shield, Oslo, Norway; stock at 60%) and underlayed below 10 mL of 17% OptiPrep/Loading Buffer gradient. The OptiPrep/Assay Buffer gradient was overlayed with 3 ml of Loading Buffer and the composite gradient centrifuged at 2,400 rpm (˜1,200 G) for 5 minutes.

The top layer of cells was removed and transferred to a centrifuge tube containing 10 mL of Cell Buffer (110 mM sucrose, 80 mM NaCl, 2 mM KCl, 10 mM HEPES, 10 mM glucose, 1 mM CaCl₂, 1 mM MgCl₂; pH 7.4), and centrifuged at 1,400 rpm (500 G) for 2 minutes. The resulting pellet was re-suspended in 10 mL of Cell Buffer. The cell suspension was filtered and the cells counted. Cell Buffer containing 3-7% OptiPrep was added to the cell suspension to obtain a cell count of 2.0 million cells/mL.

To prepare the assay using the modified Cell-Chip platform, Cell Buffer was added to both cell well 95 and waste well 94. The upstream and downstream electrode wells were loaded with Electrode Buffer (110 mM sucrose, 80 mM NaCl, 2 mM KCl, 10 mM HEPES; pH 7.4). Vacuum was applied to fill the flow channels with Cell Buffer. Cell well 95 was then emptied and a stir bar placed in each well. Waste well 94 was emptied and refilled with 100 μL of Cell Buffer containing 10-15% OptiPrep. The cell suspension (2.0 million cells/mL in Cell Buffer with 3-7% OptiPrep) was loaded into cell well 95. The modified Cell-Chip containing the assay solutions was then loaded into the Modified Caliper Personality Module.

The spinners in each of the wells were activated. After verifying spinner operation, the top and bottom fixtures of the Personality Module were attached and the assembled Modified Personality Module loaded into a Caliper 250 Detection System. The Cell-Chip was loaded with a pressure of 0 psi and moved to a dye trough. The pressure was set at 5 psi for 1 minute, followed by a pressure of −2.5 to −2.9 psi. The fluorescent probe intensity was focused and aligned. The fluid flow was set for 12 sec buffer well, and 24 sec cell well 95 with a pressure of −2.5 to −2.9 psi.

A stepped DC electric field 99 was applied to the electrodes situated in waste well 94 and electrode well 93. The applied DC electric field 99 was stepped from −500 V to −2,900 V at −250 V increments with a 100 sec dwell at each voltage level. Upon completion of the stepped ramp, the voltage was brought to 0 V for 60 sec, and the cycle repeated.

The Charge Coupled Device camera 3 was referenced to ref pd 2, with an acquisition rate of 100 Hz.

To perform the assay, the Cell Buffer containing suspended HEK-293 cells was moved along flow channel 91 through DC electric field 99. During transit through DC electric field 99, a varying transmembrane potential was induced across the membrane of the HEK-293 cells. As previously discussed, a varying transmembrane potential is known to cause activation and deactivation of voltage-regulated ion channels such as N-type calcium ion channels. The Fluo-4 AM and Fura-Red AM fluorescent probes are sensitive to the concentration of calcium ion in the surrounding medium. In this case, where the fluorescent probes were incorporated into the cytoplasm of HEK-293 cells, the intensity of the fluorescence from the probes reflected the concentration of intracellular calcium ions. Thus, activation of N-type calcium ion channels by a varying transmembrane potential was indicated by an increase in detected fluorescence. A ratio of Fluo-4 AM to Fura-Red AM was calculated from the fluorescence of each indicator probe. Fluorescence was detected in detection window 98 (cross-hatched).

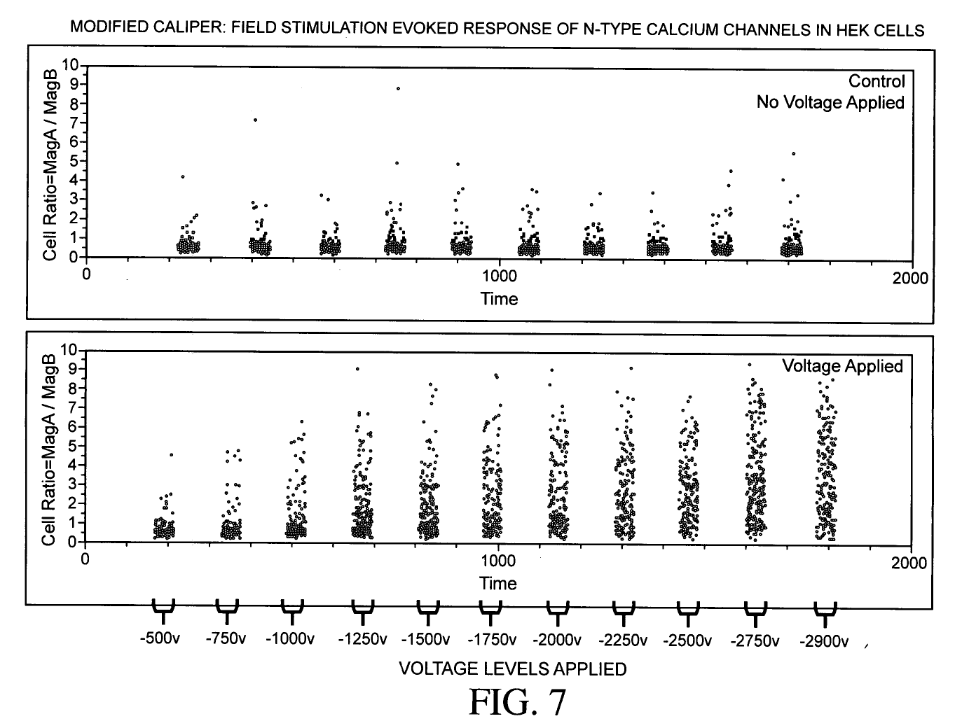

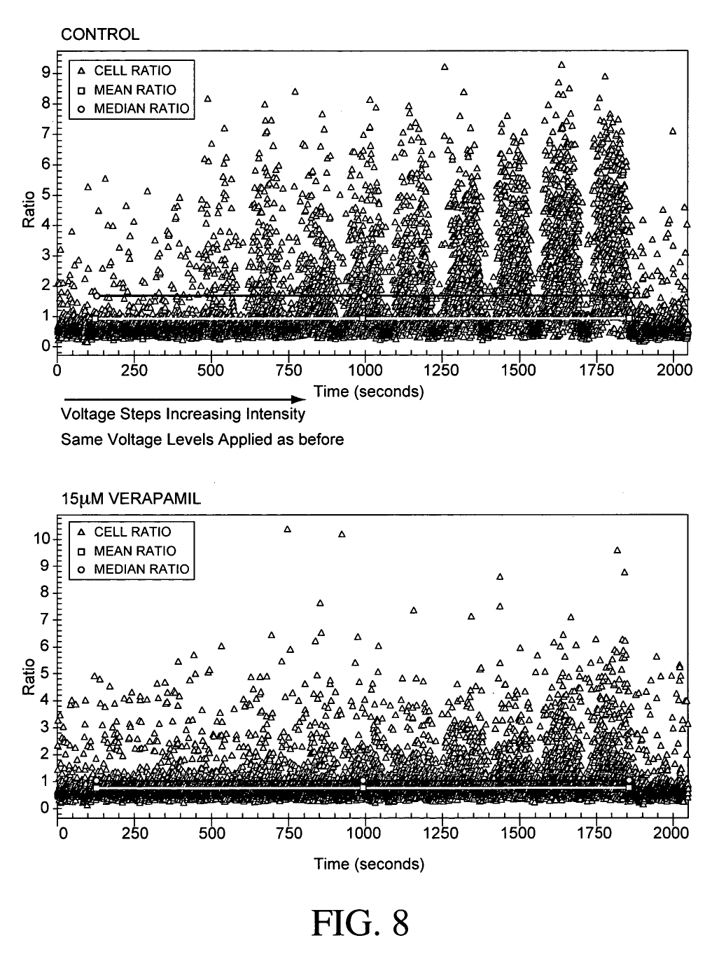

Results obtained from the above-described experiment are provided in FIG. 7 and FIG. 8. The effect of the magnitude of the applied DC electric field on the activation of N-type calcium ion channels as determined by measuring the level of intracellular calcium ions is provided in FIG. 7. As the magnitude of the DC electric field is increased, a greater amount of calcium ions enter the HEK-293 cells as shown by the increase in the average intensity of the fluorescent ratio signal. The increase in intracellular calcium ion levels with increasing applied DC electric field magnitude is consistent with activation of the N-type calcium ion channels by a varying transmembrane potential.

In FIG. 8, to verify that the influx of calcium ions resulted from activation of N-type calcium ion channels, a known antagonist of N-type calcium ion channel activity was added to the fluid containing the suspended HEK-293 cells and the assay repeated. Verapamil is one of several lipid-soluble phenylalkylamines that are known to block calcium ion channels and is useful in the treatment of supraventricular cardiac arrhythmias, angina pectoris, and hypertension. Verapamil in an amount sufficient to produce an extracellular concentration of 15 μM was added to the fluid containing the suspended HEK-293 cells. The fluid containing 15 μM verapamil and the HEK-293 cells was moved through the stepped DC electric field and the intracellular calcium ion level measured by detecting the fluorescence signal from the Fluo-4 AM and Fura-Red AM fluorescent probes incorporated into the HEK-293 cells. The results are presented in FIG. 8, where it is demonstrated that the presence of verapamil prevents influx of calcium ions into HEK-293 cells in the presence of a varying transmembrane potential induced by motion of the HEK-293 cells in the applied DC electric field.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of generating a varying potential across a membrane of a closed membrane structure in which the membrane contains a first fluid, comprising:
suspending the structure in a second fluid;
applying an electric field to the second fluid; and
moving the suspended structure in the second fluid through the electric field by non-ionic transport to induce a varying potential across the membrane.

2. A method of generating a varying potential across a membrane of a closed membrane structure in which the membrane contains a first fluid, comprising:
suspending the structure in a second fluid;
applying a DC electric field to the second fluid; and
moving the suspended structure in the second fluid through the DC electric field by non-ionic transport means to induce a varying potential across the membrane.

3. A method of generating a varying potential across a membrane of a closed membrane structure in which the membrane contains a first fluid, comprising:
suspending the structure in a second fluid;
applying a varying electric field to the second fluid; and
moving the suspended structure in the second fluid through the varying electric field by non-ionic transport to induce a varying potential across the membrane.

4. A method of generating a varying potential across a membrane of a closed membrane structure in which the membrane contains a first fluid, comprising:
suspending the structure in a second fluid;
applying an electric field to the second fluid; and
moving the second fluid so as to move the suspended structure through the electric field by non-ionic transport to induce a varying potential across the membrane.

5. A method of generating a varying potential across a membrane of a closed membrane structure in which the membrane contains a first electrically conductive fluid, comprising:
suspending the structure in a second electrically conductive fluid;
applying an electric field to the second electrically conductive fluid; and
moving the suspended structure in the second electrically conductive fluid through the electric field by non-ionic transport to induce a varying potential across the membrane.

6. The method of claim 5, wherein the electric field comprises a DC electric field.

7. The method of claim 5, wherein the electric field comprises an AC electric field.

8. The method of claim 5, wherein the electric field comprises a varying electric field.

9. The method of claim 5, wherein the electric field is selected from the group consisting of a constant electric field, a pulsed electric field, a stepped electric field, a discontinuous electric field, and a continuous waveform electric field.

10. The method of claim 5, wherein the closed membrane structure comprises at least one of a cell, an intracellular organelle, a viral envelope, and a vesicle.

11. The method of claim 10, wherein the cell is selected from the group consisting of an animal cell, an insect cell, a plant cell, and a bacterial cell.

12. The method of claim 11, wherein the animal cell is selected from the group consisting of a CHO-K1 cell, a RIN cell, and a HEK-293 cell.

13. The method of claim 10, wherein the intracellular organelle is selected from the group consisting of a mitochondria, a lysosome, a nuclear envelope, an endoplasmic reticulum, a Golgi apparatus, and a chloroplast.

14. The method of claim 5, wherein the first conductive fluid is selected from the group consisting of cytoplasm, and a physiological buffer solution.

15. The method of claim 5, wherein the second conductive fluid comprises a physiological buffer solution.

16. The method of claim 5, wherein the second conductive fluid further comprises at least one of particulates, and viscosity modifiers.

17. The method of claim 5, wherein moving the closed membrane structure comprises at least one of rotating the closed membrane structure, turning the closed membrane structure, and translating the closed membrane structure.

18. The method of claim 5, wherein non-ionic transport for moving comprises at least one of mechanical agitation, sonication, differential pressure, gradients, and gravitation.

19. The method of claim 5, wherein moving the suspended structure comprises moving the suspended structure to induce a transmembrane potential that varies spatially over the membrane expanse.

20. The method of claim 5, wherein moving the suspended structure comprises moving the suspended structure to induce a transmembrane potential that varies temporally.

21. A method of detecting the functioning of a protein of a closed membrane structure in which the membrane contains a first fluid, comprising:
suspending the structure in a second fluid;
applying an electric field to the second fluid;
moving the suspended structure in the second fluid through the electric field by non-ionic transport to induce a varying potential across the membrane sufficient to cause a change in the functioning of the protein; and
detecting indication by a probe, wherein the probe indication is produced in response to the change in the functioning of the protein.

22. A method of detecting the functioning of a protein of a closed membrane structure in which the membrane contains a first fluid, comprising:
suspending the structure in a second fluid;
applying a DC electric field to the second fluid;
moving the suspended structure in the second fluid through the DC electric field by non-ionic transport to induce a varying potential across the membrane sufficient to cause a change in the functioning of the protein; and
detecting indication by a probe, wherein the probe indication is produced in response to the change in the functioning of the protein.

23. A method of detecting the functioning of a protein of a closed membrane structure in which the membrane contains a first fluid, comprising:
suspending the structure in a second fluid;
applying a varying electric field to the second fluid;
moving the suspended structure in the second fluid through the varying electric field by non-ionic transport to induce a varying potential across the membrane sufficient to cause a change in the functioning of the protein; and
detecting indication by a probe, wherein the probe indication is produced in response to the change in the functioning of the protein.

24. A method of detecting the functioning of a protein of a closed membrane structure in which the membrane contains a first fluid, comprising:
suspending the structure in a second fluid;
applying an electric field to the second fluid;
moving the second fluid so as to move the suspended structure through the electric field by non-ionic transport to induce a varying potential across the membrane sufficient to cause a change in the functioning of the protein; and
detecting indication by a probe, wherein the probe indication is produced in response to the change in the functioning of the protein.

25. A method of detecting the functioning of a protein of a closed membrane structure in which the membrane contains a first electrically conductive fluid, comprising:
suspending the structure in a second electrically conductive fluid;
applying an electric field to the second electrically conductive fluid;
moving the suspended structure in the second electrically conductive fluid through the electric field by non-ionic transport to induce a varying potential across the membrane sufficient to cause a change in the functioning of the protein; and
detecting indication by a probe, wherein the probe indication is produced in response to the change in the functioning of the protein.

26. The method of claim 25, wherein the electric field comprises a DC electric field.

27. The method of claim 25, wherein the electric field comprises an AC electric field.

28. The method of claim 25, wherein the electric field comprises a varying electric field.

29. The method of claim 25, wherein the electric field is selected from the group consisting of a constant electric field, a pulsed electric field, a stepped electric field, a discontinuous electric field, and a continuous waveform electric field.

30. The method of claim 25, wherein the closed membrane structure is selected from the group consisting of at least one of a cell, an intracellular organelle, a viral envelope, and a vesicle.

31. The method of claim 30, wherein the cell is selected from the group consisting of an animal cell, an insect cell, a plant cell, and a bacterial cell.

32. The method of claim 31, wherein the animal cell is selected from the group consisting of a CHO-K1 cell, a RIN cell, and a HEK-293 cell.

33. The method of claim 30, wherein the intracellular organelle is selected from the group consisting of a mitochondria, a lysosome, a nuclear envelope, an endoplasmic reticulum, a Golgi apparatus, and a chloroplast.

34. The method of claim 25, wherein the first conductive fluid is selected from the group consisting of cytoplasm, and a physiological buffer solution.

35. The method of claim 25, wherein the second conductive fluid comprises a physiological buffer solution.

36. The method of claim 25, wherein the second conductive fluid further comprises at least one of particulates, and viscosity modifiers.

37. The method of claim 25, wherein moving the closed membrane structure comprises at least one of turning the closed membrane structure, rotating the closed membrane structure, and translating the closed membrane structure.

38. The method of claim 25, wherein moving by non-ionic transport comprises at least one of mechanical agitation, sonication, differential pressure, gradients, and gravitation.

39. The method of claim 25, wherein moving the suspended structure comprises moving the suspended structure to induce a transmembrane potential that varies spatially over the membrane expanse.

40. The method of claim 25, wherein moving the suspended structure comprises moving the suspended structure to induce a transmembrane potential that varies temporally.

41. The method of claim 25, wherein the probe is selected from the group consisting of an endogenous probe, an exogenous probe, and combinations thereof.

42. The method of claim 25, wherein the probe indication is sensitive to at least one of the expression of a compound, the concentration of a compound, the presence of a compound, the translocation of a compound, the flux of a compound, the conformation of a compound, the activity of a compound, the environment of a compound, and the structure of a compound.

43. The method of claim 25, wherein the probe is situated in at least one of the first fluid, the second fluid, the exterior expanse of the membrane, the interior expanse of the membrane, and within the membrane.

44. The method of claim 25, wherein detecting indication by a probe comprises detecting at least fluorescence, luminescence, radiation, electrical current, atomic absorption spectroscopy, and mass spectroscopy.

45. The method of claim 25, wherein the probe is selected from the group consisting of an ion probe, a membrane potential probe, a probe sensitive to phosphorylation, a probe sensitive to enzymatic activity, and a probe sensitive to translocation between cellular compartments.

46. The method of claim 45, wherein the ion probe is selected from the group consisting of sodium ion probes, potassium ion probes, calcium ion probes, chloride ion probes, thallium ion probes, rubidium ion probes, lithium ion probes, hydrogen ion probes, bicarbonate ion probes, and nitrate ion probes.

47. The method of claim 45, wherein the membrane potential probe is selected from the group consisting of an oxonol derivative dye, a rhodamine derivative dye, a styryl derivative dye, a fluorescence resonance energy transfer probe, a GFP-type mutant, and a photoprotein.

48. The method of claim 25, wherein the membrane is selected from the group consisting of an extracellular membrane, an intracellular membrane, a vesicle membrane, and a synthetic membrane.

49. The method of claim 25, wherein the protein is selected from the group consisting of a protein located on the exterior expanse of the closed membrane structure, a protein located on the interior expanse of the closed membrane structure, a transmembrane protein, and an embedded protein.

50. The method of claim 25, wherein the protein is selected from the group consisting of a single protein, a collection of proteins, and a protein complex.

51. The method of claim 25, wherein the protein complex comprises more than one protein wherein the more than one protein may be the same or different.

52. The method of claim 25, wherein the protein is selected from the group consisting of an endogenously expressed protein, and an exogenously expressed protein.

53. The method of claim 25, wherein the protein is selected from the group consisting of an ion channel, an ion transporter, a receptor, a GPCR/7TM, a kinase, a phosphatase, and a protease.

54. The method of claim 53, wherein the ion channel is selected from the group consisting of a sodium ion channel, a potassium ion channel, a calcium ion channel, and a chloride ion channel.

55. The method of claim 53, wherein the ion channel is selected from the group consisting of a voltage-gated ion channel, a ligand-gated ion channel, and a mechanically-gated ion channel.

56. The method of claim 53, wherein the ion transporter is selected from the group consisting of an ion transporter which utilizes adenosine triphosphate energy directly, and an ion transporter which utilizes adenosine triphosphate energy indirectly.

57. A method of detecting the effect of an agent on the functioning of a protein of a closed membrane structure in which the membrane contains a first fluid, comprising:
suspending the structure in a second fluid;
applying an electric field to the second fluid;
adding the agent to the second fluid;
moving the suspended structure in the second fluid through the electric field by non-ionic transport to induce a varying potential across the membrane; and
detecting indication by a probe, wherein a probe indication is produced in response to the change in the functioning of the protein.

58. The method of claim 57, wherein the agent is selected from the group consisting of a compound, a peptide, and a protein.

59. The method of claim 57, wherein the electric field comprises a DC electric field.

60. The method of claim 57, wherein the electric field comprises an AC electric field.

61. The method of claim 57, wherein the electric field comprises a varying electric field.

62. The method of claim 57, wherein the electric field is selected from the group consisting of a constant electric field, a pulsed electric field, a stepped electric field, a discontinuous electric field, and a continuous waveform electric field.

63. The method of claim 57, wherein the closed membrane structure is selected from the group consisting of at least one of a cell, an intracellular organelle, a viral envelope, and a vesicle.

64. The method of claim 63, wherein the cell is selected from the group consisting of an animal cell, an insect cell, a plant cell, and a bacterial cell.

65. The method of claim 63, wherein the animal cell is selected from the group consisting of a CHO-K1 cell, a RIN cell, and a HEK-293 cell.

66. The method of claim 57, wherein the intracellular organelle is selected from the group consisting of a mitochondria, a lysosome, a nuclear envelope, an endoplasmic reticulum, a Golgi apparatus, and a chloroplast.

67. The method of claim 57, wherein the first conductive fluid is selected from the group consisting of cytoplasm, and a physiological buffer solution.

68. The method of claim 57, wherein the second conductive fluid comprises a physiological buffer solution.

69. The method of claim 57, wherein the second conductive fluid further comprises at least one of particulates, and viscosity modifiers.

70. The method of claim 57, wherein moving the closed membrane structure comprises at least one of turning the closed membrane structure, rotating the closed membrane structure, and translating the closed membrane structure.

71. The method of claim 57, wherein moving by non-ionic transport comprises at least one of mechanical agitation, sonication, differential pressure, gradients, and gravitation.

72. The method of claim 57, wherein moving the suspended structure comprises moving the suspended structure to induce a transmembrane potential that varies spatially over the membrane expanse.

73. The method of claim 57, wherein moving the suspended structure comprises moving the suspended structure to induce a transmembrane potential that varies temporally.

74. The method of claim 57, wherein the probe is selected from the group consisting of an endogenous probe, an exogenous probe, and combinations thereof.

75. The method of claim 57, wherein the probe indication is sensitive to at least one of the expression of a compound, the concentration of a compound, the presence of a compound, the translocation of a compound, the flux of a compound, the conformation of a compound, the activity of a compound, the environment of a compound, and the structure of a compound.

76. The method of claim 57, wherein the probe is situated in at least one of the first fluid, the second fluid, the exterior expanse of the membrane, the interior expanse of the membrane, and within the membrane.

77. The method of claim 57, wherein detecting indication by a probe comprises detecting at least fluorescence, luminescence, radiation, electrical current, atomic absorption spectroscopy, and mass spectroscopy.

78. The method of claim 57, wherein the probe is selected from the group consisting of an ion probe, a membrane potential probe, a probe sensitive to intracellular phosphorylation, and a probe sensitive to translocation between cellular compartments.

79. The method of claim 78, wherein the ion probe is selected from the group consisting of sodium ion probes, potassium ion probes, calcium ion probes, chloride ion probes, thallium ion probes, rubidium ion probes, lithium ion probes, hydrogen ion probes, bicarbonate ion probes, and nitrate ion probes.

80. The method of claim 78, wherein the membrane potential probe is selected from the group consisting of an oxonol derivative dye, a rhodamine derivative dye, a styryl derivative dye, a fluorescence resonance energy transfer probe, a GFP-type mutant, and a photoprotein.

81. The method of claim 57, wherein the membrane is selected from the group consisting of an extracellular membrane, an intracellular membrane, a vesicle membrane, and a synthetic membrane.

82. The method of claim 57, wherein the protein is selected from the group consisting of a protein located on the exterior expanse of the closed membrane structure, a protein located on the interior expanse of the closed membrane structure, a transmembrane protein, and an embedded protein.

83. The method of claim 57, wherein the protein is selected from the group consisting of a single protein, a collection of proteins, and a protein complex.

84. The method of claim 57, wherein the protein complex comprises more than one protein wherein the more than one protein may be the same or different.

85. The method of claim 57, wherein the protein is selected from the group consisting of an endogenously expressed protein, and an exogenously expressed protein.

86. The method of claim 57, wherein the protein is selected from the group consisting of an ion channel, an ion transporter, a receptor, a GPCR/7TM, a kinase, a phosphatase, and a protease.

87. The method of claim 86, wherein the ion channel is selected from the group consisting of a sodium ion channel, a potassium ion channel, a calcium ion channel, and a chloride ion channel.

88. The method of claim 86, wherein the ion channel is selected from the group consisting of a voltage-gated ion channel, a ligand-gated ion channel, and a mechanically-gated ion channel.

89. The method of claim 86, wherein the ion transporter is selected from the group consisting of an ion transporter which utilizes adenosine triphosphate energy directly, and an ion transporter which utilizes adenosine triphosphate energy indirectly.

90. An apparatus for generating a varying potential across a membrane of a closed membrane structure in which the membrane contains a first fluid, wherein the structure is suspended in a second fluid, comprising:
a fluid chamber containing the second fluid;
electrodes applying an electric field to at least a part of the fluid chamber; and
non-ionic transport means for moving the suspended structure in the second fluid through the electric field to induce a varying potential across the membrane.

91. An apparatus for generating a varying potential across a membrane of a closed membrane structure in which the membrane contains a first fluid, wherein the structure is suspended in a second fluid, comprising:
a first well containing the second fluid;
a second well;
a third well;
a fluid channel fluidly connecting the first well, the second well, and the third well;
electrodes for generating an electric field electrically connected to the fluid in the second well and in the third well;
a voltage source electrically connected to the electrodes;
a spinner in the first well;
a stirrer mechanism positioned under the first well; and
a detector for detecting indication by a probe.

92. An apparatus for detecting the functioning of a protein of a closed membrane structure in which the membrane contains a first fluid, wherein the structure is suspended in a second fluid, comprising:
a fluid chamber containing the second fluid;
electrodes applying an electric field to at least a part of the fluid chamber;
non-ionic transport means for moving the suspended structure in the second fluid through the electric field to induce a varying potential across the membrane sufficient to cause a change in the functioning of the protein; and
a detector for detecting indication by a probe, wherein the probe indication is produced in response to the change in the functioning of the protein.

93. An apparatus for detecting the effect of an agent on the functioning of a protein of a closed membrane structure in which the membrane contains a first fluid, wherein the structure is suspended in a second fluid, comprising:
a fluid chamber containing the second fluid;
electrodes applying an electric field to at least a part of the fluid chamber;
non-ionic transport means for moving the suspended structure in the second fluid through the electric field to induce a varying potential across the membrane; and
a detector for detecting indication by a probe, wherein the probe indication is produced in response to a change in the functioning of the protein.

94. The apparatus of claim 93, wherein the fluid chamber is a flow channel.

95. The apparatus of claim 93, wherein the fluid chamber comprises at least a part exposed to the electric field and a part for detecting indication by the probe.

96. The apparatus of claim 93, further comprising a voltage source.

97. The apparatus of claim 93, further comprising at least two electrodes connected to the voltage source.

98. The apparatus of claim 93, wherein the apparatus is a microfluidic apparatus.

99. The apparatus of claim 93, wherein the detector is selected from the group consisting of a fluorescence detector, a luminescence detector, a radiation detector, a current detector, atomic absorption spectrometer, and mass spectrometer.

100. An apparatus for generating a varying potential across membranes of closed membrane structures in which the membranes contain a first fluid, wherein the structures are suspended in a second fluid, comprising:
a plurality of fluid chambers containing the second fluid;
at least two electrodes applying an electric field to at least a part of each of the plurality of fluid chambers; and
at least one non-ionic transport means for moving the suspended structures in the second fluid through the at least one electric field applied to at least a part of each of the plurality of fluid chambers to induce a varying potential across the membranes.

101. The apparatus of claim 100, wherein the at least two electrodes apply the same magnitude electric field to at least a part of each of the plurality of fluid chambers.

102. The apparatus of claim 100, wherein the at least two electrodes apply a different magnitude electric field to at least a part of each of the plurality of fluid chambers.

103. The apparatus of claim 100, wherein more than one electric field is applied to each of the plurality of fluid chambers.

104. The apparatus of claim 103, wherein the more than one electric field comprises a different magnitude electric field.

105. An apparatus for detecting the functioning of proteins of closed membrane structures in which the membranes contain a first fluid, wherein the structures are suspended in a second fluid, comprising:
a plurality of fluid chambers containing the second fluid;
at least two electrodes applying an electric field to at least a part of each of the plurality of fluid chambers;
at least one non-ionic transport means for moving the suspended structures in the second fluid through the at least one electric field applied to at least a part of each of the plurality of fluid chambers to induce a varying potential across the membrane sufficient to cause a change in the functioning of the protein; and
at least one detector for detecting indication by a probe, wherein the probe indication is produced in response to the change in the functioning of the proteins.

106. The apparatus of claim 105, wherein the at least two electrodes apply the same magnitude electric field to at least a part of each of the plurality of fluid chambers.

107. The apparatus of claim 105, wherein the at least two electrodes apply a different magnitude electric field to at least a part of each of the plurality of fluid chambers.

108. The apparatus of claim 105, wherein the more than one electric field comprises a different magnitude electric field.

109. The apparatus of claim 105, wherein the more than one detector comprises a different detector.

110. An apparatus for detecting the effect of an agent on the functioning of proteins of closed membrane structures in which the membranes contain a first fluid, wherein the structures are suspended in a second fluid, comprising:
a plurality of fluid chambers containing the second fluid;
at least one two electrodes applying an electric field to at least a part of each of the plurality of fluid chambers;
at least one non-ionic transport means for moving the suspended structures in the second fluid through the at least one electric field applied to at least a part of each of the plurality of fluid chambers to induce a varying potential across the membranes; and
at least one detector for detecting indication by a probe from each of the plurality of fluid chambers, wherein the probe indication is produced in response to a change in the functioning of the proteins.
